(19) United States
(12) Patent Application Publication
(10) Pub. No.: US 2014/0142620 A1
(43) Pub. Date: May 22, 2014

(54) DEGRADABLE BALLOON DEVICE AND METHOD FOR CLOSURE OF OPENINGS IN A TISSUE WALL

(71) Applicant: COOK MEDICAL TECHNOLOGIES LLC, BLOOMINGTON, IN (US)

(72) Inventors: Benjamin Marchi, WEST LAFAYETTE, IN (US); JARIN KRATZBERG, LAFAYETTE, IN (US)

(21) Appl. No.: 14/064,668

(22) Filed: Oct. 28, 2013

Related U.S. Application Data
(60) Provisional application No. 61/727,958, filed on Nov. 19, 2012.

(51) Int. Cl. A61B 17/00

(52) U.S. Cl. CPC A61B 17/0057
USPC 606/213

(57) ABSTRACT

This discloses a device for closure of an opening in a medical patient's subcutaneous tissue wall. The devices disclosed included a catheter having one or more inflatable, bio-degradable balloon and one or more sheets of ECM material loaded on and around said catheter. Upon inflation of the one or more balloons the sheet(s) of ECM material are held against the tissue wall by the balloon(s). Surgical methods using the device are also disclosed.

### CROSS-REFERENCE TO RELATED APPLICATIONS

This application claims the benefit of U.S. Provisional Application No. 61/727,958, filed Nov. 19, 2012, which is hereby incorporated by reference in its entirety.

### BACKGROUND

The present invention relates to closure of openings in tissue walls percutaneously, and more specifically to methods and devices therefor with one or more balloons.

Percutaneous access provides minimally invasive access to subcutaneous tissues walls, such as for example vascular access without excessive cut downs or other invasive techniques. Access is acquired by a needle puncture through the skin into the desired vessel. A guide wire is then inserted into the vessel through the access site. The initial puncture is expanded through progressive dilation over the guide wire; the access site is dilated such that the desired endovascular device can be inserted into the vessel. Once the procedure is complete, the delivery system of the endovascular device is removed through the access site. However, once the delivery system is withdrawn from the vessel a lesion remains. The device described herein provides temporary hemostatis around the access site (specifically the lesion in the vessel wall), and promotes healing of the native vessel with small intestine submucosa (SIS).

Current percutaneous closure devices are typically limited to use in peripheral access cases (access site size typically less than or equal to 10 Fr), and some require permanent implantation; direct pressure on the access site is also used to provide hemostasis. Thus, there is a need for improvement in this field.

### SUMMARY

The present invention is defined by the claims. As merely a summary, this may include a device for closure of an opening in a medical patient's subcutaneous tissue wall. The device may include a catheter having one or more inflatable, bio-degradable balloons. It may also include one or more sheets of extracellular matrix (ECM) material loaded on and around said catheter, wherein upon inflation of the one or more balloons the sheet(s) of ECM material are held against the tissue wall by the balloon(s).

The present invention may also include surgical insertion methods of the various devices shown and described.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a perspective view of one example of the present invention.

FIGS. 2A-2F are side cut away views of the insertion of the device of FIG. 1.

FIG. 3 is partial cut away view of one example of a catheter of the device of FIG. 1.

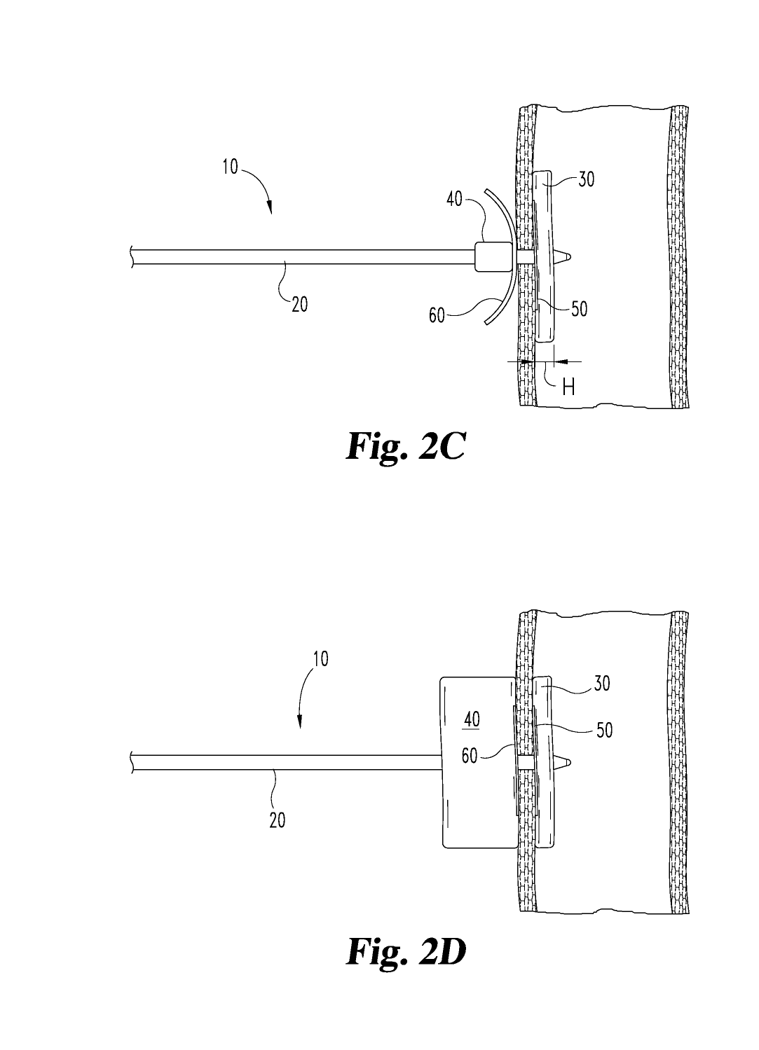

FIG. 4 is a perspective view of one example of a sheet of ECM material shown in isolation.

FIG. 5 is a side cross sectional view of another example of two sheets (joined) of ECM material shown in isolation.

FIG. 6 is partial cut away perspective detail of another example of the device of FIG. 1.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

Percutaneous access provides minimally invasive vascular access without excessive cut downs or other invasive techniques. Access is acquired by a needle puncture through the skin into the desired vessel. A guide wire is then inserted into the vessel through the access site. The initial puncture is expanded through progressive dilation over the guide wire; the access site is dilated such that the desired endovascular device can be inserted into the vessel. Once the procedure is complete, the delivery system of the endovascular device is removed through the access site. However, once the delivery system is withdrawn from the vessel a lesion remains. The device described herein provides temporary hemostatis around the access site (specifically the lesion in the vessel wall), and promotes healing of the native vessel with SIS.

Current percutaneous closure devices are limited to use in peripheral access cases (access site size typically less than or equal to 10 Fr), and some require permanent implantation; direct pressure on the access site is also used to provide hemostasis. The proposed device allows for closure from large French size delivery systems typically used in endovascular aneurysmal and abdominal arterial repairs. This device also performs the closure with no long term implantation. The device design will target closure of 6-24 Fr percutaneous access.

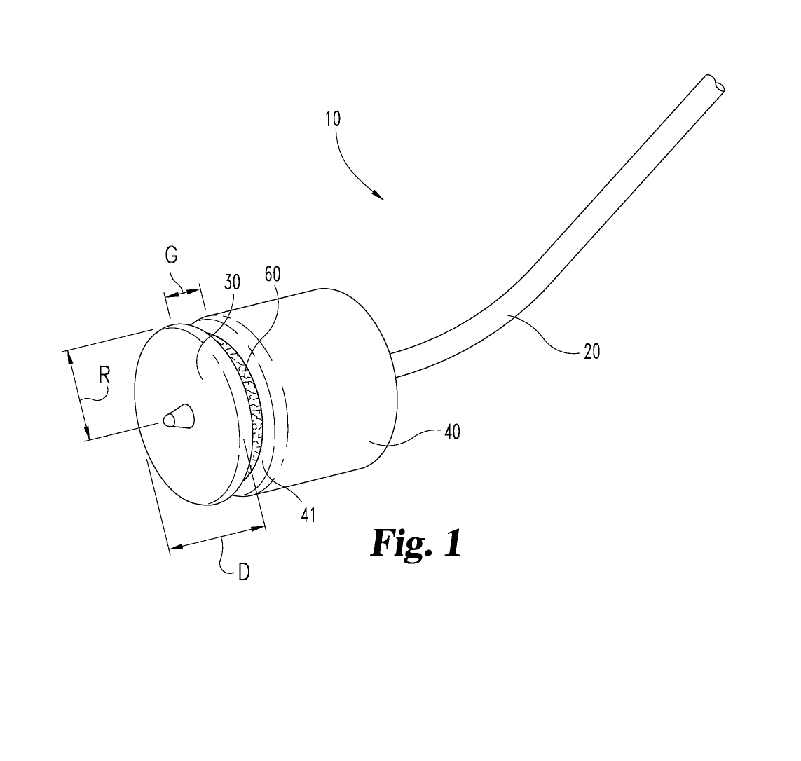

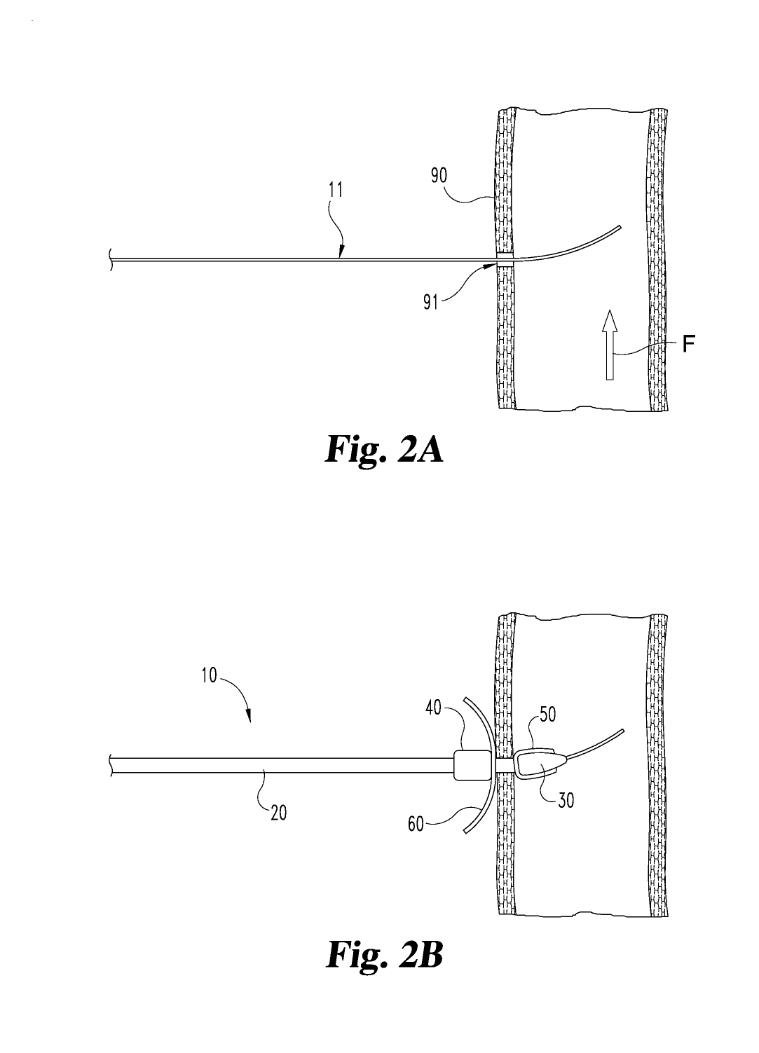

The proposed design consists of a balloon catheter, knot pusher, and a decoupler. These components are housed typically in a 12 Fr sheath, although other sizes may be used. There are two balloons on the catheter. The distal balloon is situated near the tip of the catheter. This balloon is disk like in shape (FIGS. 1 and 2); the balloon has a diameter larger than the lesion (approximately 3 mm greater than the outer diameter of the original device delivery system), and a height such that the protrusion into the vessel does not negatively affect the blood flow, namely occupy less than 40% of the cross-sectional area of the vessel's lumen. This geometry promotes good sealing of the lesion, as well as protection against migration. The proximal balloon also has a cylindrical shape, and has the same diameter as the distal balloon. The height of the proximal balloon is 2-3 times greater than the distal balloon. The balloons are positioned on the catheter such that there is a 1-2 mm gap between the expanded balloon profiles. Hemostasis is achieved when the proximal balloon is expanded. The size of the gap between the balloons, along with the balloon geometry, promotes sealing against both sides of the vessel. An SIS disk is fixed to both the proximal surface of the distal balloon and the distal surface of the proximal balloon (FIGS. 1 and 2). When the balloons are expanded the SIS will be in contact with both sides of the vessel wall to promote healing of the native vessel. Both the balloons and the catheter will be made of biodegradable/bioabsorbable polymer.

A pre-tied sliding and self-locking knot will be looped over the catheter proximal to the proximal balloon. The post of the knot will be threaded through the lumen of a stiff cannula pusher. The pusher will be used to tighten the knot onto the catheter. The knot will be tied with an absorbable suture. The knot pusher will extend past the sheath, and it will be manipulated by the physician to achieve proper knot positioning. When a decoupler is employed, it will cut the catheter proximal to the knot. A possible decoupler consists of coiled wires which, when current is applied, fuse and sever the catheter stem.

The proposed device allows for closure from large French size delivery systems typically used in endovascular aneurysmal and abdominal arterial repairs. This device also performs the closure with no long term implantation. The device design will target closure of typically 6-24 Fr, and preferably 12-24 Fr, percutaneous access.

The term “bio-degradable” means able to be broken down and absorbed in, resorbed and/or passed from the human body.

The term “central hole” means an opening (round, slit(s), rectangular or any other shape) in a sheet or other member that is at least partially within about the central two-thirds of the shape.

The term “closing a lumen” means reducing its cross section at one or more points sufficient to substantially restrict fluid flow through the lumen. This may be done with one or more knot, noose, check valve, plug, flap and/or other structure.

The term “cutting off” means fully or substantially severing one part so it may be separated from another.

The term “depth” means the average distance of an element taken in a direction proximal to distal.

The term “distal” means, relative to a reference, toward the doctor or other operator of the device; normally the opposite of “proximal”.

The term “ECM material” means material harvested from a (previously) living animal which is an extracellular matrix, typically including collagen. This includes without limitation material harvested from the walls of animal intestine, bladder, liver, and stomach.

The term “loaded” means placed into position prior to insertion into a patient.

The term “proximal” means, relative to a reference, away from the doctor or other operator of the device; normally the opposite of “distal”.

The term “radius” means the average distance of an element taken in a direction from the center to the perimeter.

The term “sheet” means a generally flat member which is thinner in depth than in the other dimensions and is at least somewhat flexible. It may be single layer, multi-layer, formed, cut, pressed, molded, porous, non-porous, and/or otherwise. A sheet may be round (such as a round disk), rectangular, square, elliptical, hexagonal, octagonal, oblong, irregular or any other shape.

The term “small intestine submucosa” means material (sheet, solid shape, powder, liquid or otherwise) which is entirely or partially made up of portions of the submucosal layer of an animal's small intestines.

The term “subcutaneous tissue wall” means any tissue wall of a patient beneath the skin, including without limitation blood vessel walls.

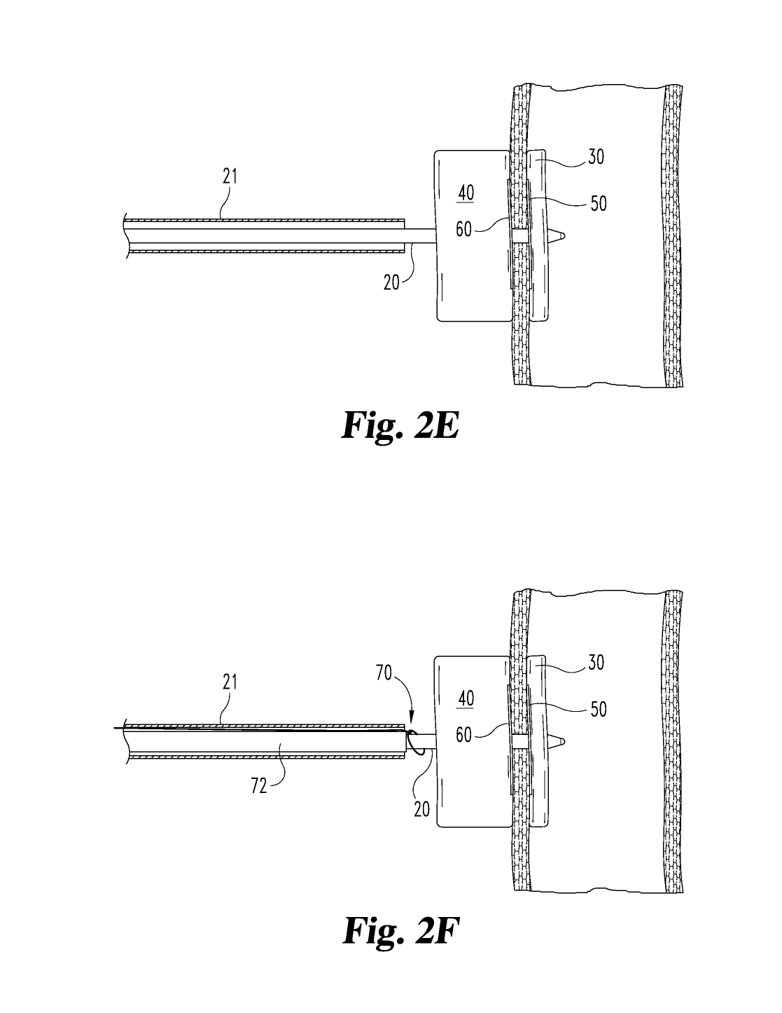

The proposed device 10 may comprise a balloon catheter 20, an optional knot pusher 72 (see FIG. 2F), and an optional decoupler. Or, the balloons and sheets may be mounted and dismountable from the catheter. These components are typically housed in a sheath 21, at least during part of installation, such as for example a 12 Fr sheath. The catheter may have lumen L advance-able over a wire guide 11. There are normally two balloons 30 and 40 on the catheter. The distal balloon 30 is situated at or near the tip of the catheter. This balloon, when inflated, may be disk like in shape (see FIGS. 1 and 2C); the balloon has a diameter D (twice radius R)(see FIG. 1) larger than the lesion or opening 91 in tissue wall 90 (FIG. 2A) (the diameter typically approximately 3 mm greater or more than the outer diameter of the original device delivery system), and a depth, (also referred to herein as height H) (see FIGS. 2C and 2E) such that the protrusion into the vessel does not negatively affect any fluid flow, such as for example blood flow F (see FIG. 2A). This geometry promotes good sealing of the lesion, as well as protection against migration. The proximal balloon 40, when inflated, also may have a cylindrical (or other) shape, and typically has about the same diameter as the distal balloon 30. The depth of the proximal balloon may be smaller, the same as or greater, for example 2-3 times greater, than the distal balloon. Normally it is greater as illustrated. The balloons are positioned on the catheter such that there is a 1-2 mm gap G (see FIGS. 1 and 2D) between the expanded balloon profiles. Hemostasis or other closure may be achieved when the proximal balloon is expanded. The size of the gap between the balloons, along with the balloon geometry, promotes sealing against both sides of the vessel. An extracellular matrix (ECM) material sheet 50, 60, in the shape of a disk or otherwise, is loaded on, and optionally fixed to, one or both of the proximal surface of the distal balloon and the distal surface 41 of the proximal balloon (FIGS. 1 and 2). The sheet may be any shape, with a round disk example shown in FIG. 4 with a central opening 51 to facilitate loading onto the catheter. When the balloons are expanded the ECM will be in contact with both sides of the vessel wall 90 to promote healing of the native vessel. Both the balloons and the catheter will be made of bio-degradable material, including bio-absorbable material, such as a bio-degradable polymer.

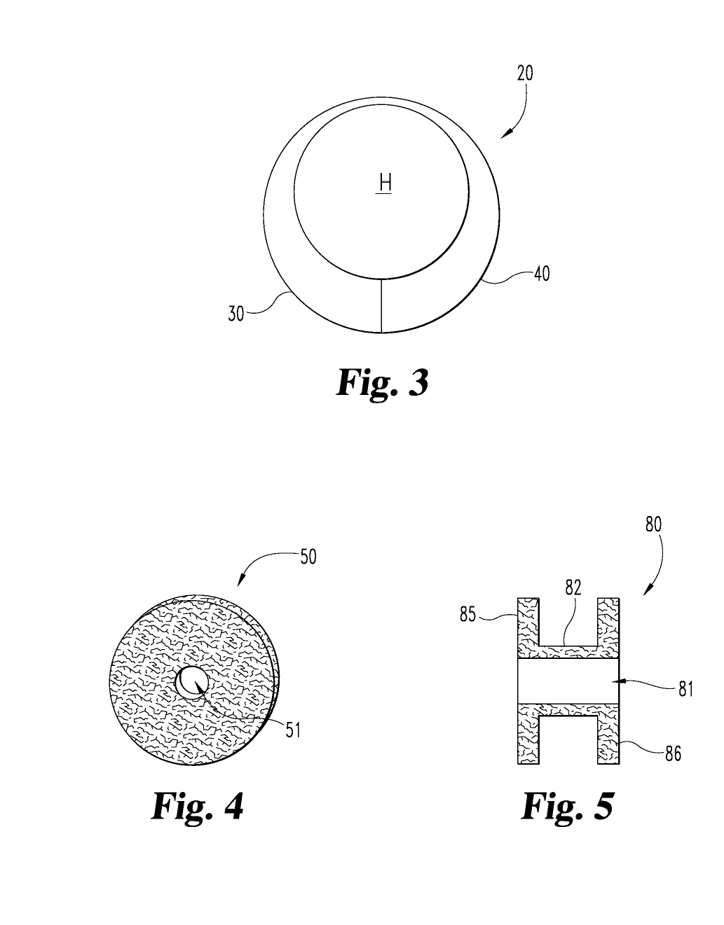

An optional type of alternative of a sheet of FIG. 4 is shown in FIG. 5, a side cross sectional view. There, implant 80 comprises at least two ECM material sheets 85, 86 joined by connection 82. Connection 82 may be ECM material or other bio-degradable material. Lumen 81 may receive the catheter through it so as to provide a balloon on the distal side of implant 80. A pre-tied sliding and self-locking knot 70 may be looped over the catheter proximal to the proximal balloon. The post of the knot may be threaded through the lumen of a stiff cannula pusher 72. The pusher will be used to tighten the knot onto the catheter. The knot may be tied with an absorbable suture. The knot pusher can extend past the sheath and be manipulated by the physician to achieve proper knot positioning. It may be tightened for closing a lumen, such as to close one or more lumens in the catheter maintaining balloons 30 and/or 40 inflated.

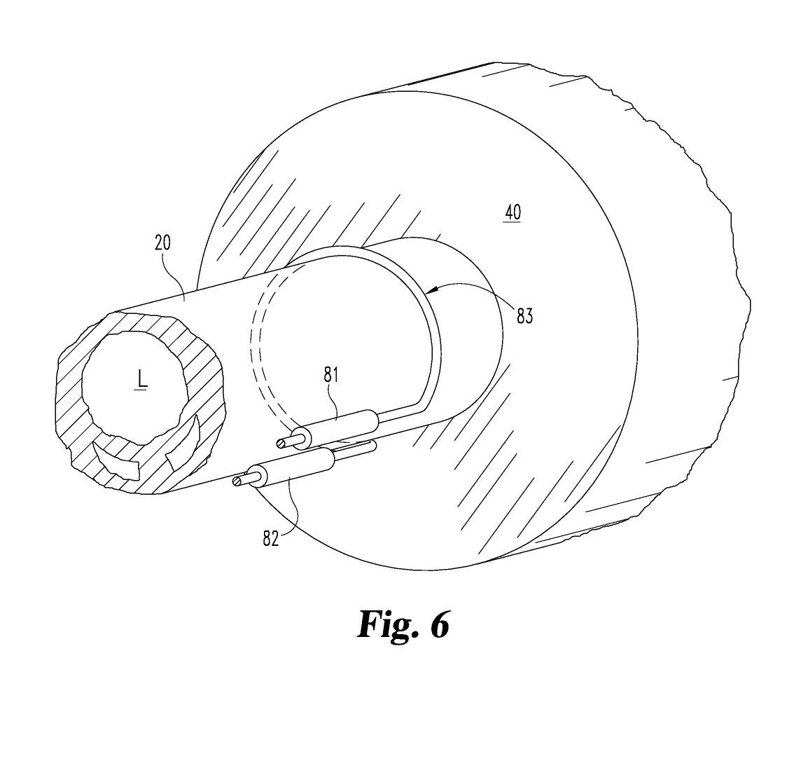

A decoupler may be used to cut the catheter proximal to the knot. A possible decoupler consists of coiled wire(s) 81, 82, 83 (see FIG. 6) which, when electric current is applied, provide for cutting the catheter stem, preferably proximal to the elements that close the lumens.

The following is a summary of one example that can be used for the delivery procedure for the devices disclosed herein:

1. Remove delivery system of the last endovascular device used in the repair, while maintaining vessel access with the guide wire.

2. Prep the proposed closure device's balloons, and flush the catheter with saline.

3. Advance the device until into the desired vessel.

4. Expand the distal balloon while inside the vessel. Then pull back on the sheath to verify that the inflated balloon can provide adequate closure of the lesion.

5. While maintaining tension against the inner side of the vessel wall, inflate the proximal balloon. There should now be successful closure of the lesion or opening. Retract the guide wire.

6. Advance the pusher to tighten the knot.

7. Once the knot is tight, engage the decoupler to cut the catheter and extra suture.

8. Remove the remains of the severed delivery system from the access site.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A device for closure of an opening in a medical patient's subcutaneous tissue wall, including:
a catheter having a first inflatable, bio-degradable balloon;
a sheet of ECM material loaded on and around said catheter, wherein upon inflation of said first balloon said sheet of ECM material is located proximal of said first balloon and is held against the tissue wall by said first balloon.

2. The device of claim 1 and further including:
a second inflatable, bio-degradable balloon of said catheter located proximal of said first balloon;
a second sheet of ECM material loaded on and around said catheter, wherein upon inflation of said second balloon said second sheet of ECM material is located distal of said second balloon and is held against the tissue wall by said second balloon.

3. The device of claim 1, wherein said tissue wall is locatable between said first and second sheets of ECM material, and wherein said sheets of ECM material are pressed together by said first and second balloons.

4. The device of claim 1 and further including:
means for closing a lumen of said catheter after one or more of the balloons of said catheter are inflated.

5. The device of claim 1 and further comprising means for cutting off said catheter subcutaneously and distally of each of the catheter's balloons.

6. The device of claim 1, wherein at least one sheet of said ECM material is generally round in shape with a central hole closely conforming to the outer diameter of said catheter.

7. The device of claim 1, wherein at least one sheet of ECM material comprises small intestine submucosa.

8. The device of claim 1, wherein at least a portion of said catheter adjacent said one or more balloons is bio-degradable.

9. The device of claim 5, wherein said means for cutting comprises a wire in said catheter to conduct electrical current to generate cutting heat around a circumference of said catheter to weaken or severe the catheter subcutaneously.

10. The device of claim 4, wherein said means for closing comprises a cord formed in a noose to cinch said catheter.

11. The device of claim 1, wherein said first balloon is generally disc shaped, having a depth less than a radius thereof.

12. A method of closure of an opening in a medical patient's subcutaneous tissue wall comprising the act of inserting in a medical patient a device of claim 1.

13. A method for closure of an opening in a medical patient's subcutaneous tissue wall, including the acts of:
Inserting a catheter having: (a) a first inflatable, bio-degradable balloon and a sheet of ECM material loaded proximal of said first balloon on and around said catheter; and, (b) a second inflatable, bio-degradable balloon of said catheter located proximal of said first balloon and a second sheet of ECM material loaded distal of said second balloon on and around said catheter with said first balloon and said first sheet of ECM material through the opening in the tissue wall;
Inflating said first balloon and second balloon with said first and said second sheets of ECM material held on opposite sides of the tissue wall.

14. The device of claim 2, wherein said tissue wall is locatable between said first and second sheets of ECM material, and wherein said sheets of ECM material are pressed together by said first and second balloons.

15. The device of claim 14 and further including:
means for closing a lumen of said catheter after one or more of the balloons of said catheter are inflated.

16. The device of claim 15 and further comprising means for cutting off said catheter subcutaneously and distally of each of the catheter's balloons.

17. The device of claim 16, wherein at least one sheet of said ECM material is generally round in shape with a central hole closely conforming to the outer diameter of said catheter.

18. The device of claim 17, wherein at least one sheet of ECM material comprises small intestine submucosa.

19. The device of claim 18, wherein at least a portion of said catheter adjacent said one or more balloons is bio-degradable.

20. The device of claim 19, wherein said means for cutting comprises a wire in said catheter to conduct electrical current to generate cutting heat around a circumference of said catheter to weaken or severe the catheter subcutaneously.
